(19) United States
(12) Patent Application Publication
(10) Pub. No.: US 2017/0360565 A1
(43) Pub. Date: Dec. 21, 2017

(54) METHOD FOR PRODUCING AN ATTACHMENT PIECE OF A COVER DEVICE FOR A BONE DEFECT SITE, AND DEVICE FOR COVERING AND/OR RECONSTRUCTING A BONE DEFECT SITE

(71) Applicant: ReOss GmbH, Filderstadt (DE)

(72) Inventor: Marcus SEILER, Stuttgart (DE)

(73) Assignee: ReOss GmbH, Filderstadt (DE)

(21) Appl. No.: 15/542,959

(22) PCT Filed: Jan. 11, 2016
(86) PCT No.: PCT/DE2016/000016

§ 371 Date: Jul. 12, 2017

(30) Foreign Application Priority Data
Jan. 13, 2015 (DE) 10 2015 000 496.4
May 8, 2015 (DE) 10 2015 006 153.4

(51) Int. Cl. A61F 2/28; G06F 19/00; A61L 31/06; A61L 31/14; A61L 31/16; A61C 8/00; A61C 8/02; A61F 2/30

(52) U.S. Cl. CPC A61F 2/2846; A61C 8/008; G06F 19/325; A61C 8/0006; A61L 31/06; A61L 31/146; A61L 31/148; A61L 31/16; A61F 2002/285; A61F 2002/2835; A61F 2002/30034; A61F 2002/30062; A61F 2002/30561; A61F 2002/30784; A61F 2002/30838; A61F 2002/3093; A61L 2300/412; A61L 2400/18

(57) ABSTRACT

A process for manufacturing a cap (4) which has at least one nominal breaking point (16) of a covering device for a bone defect site (2) and a device (1) for covering and/or reconstructing a bone defect site (2) are proposed, wherein through comparing a first data set which represents the affected bone defect site (2) in the actual condition with a second data set which represents the nominal condition of a regenerated bone at the bone defect site (2), wherein the second data set has been calculated or recorded at a time at which the bone at the site now to be regenerated was still a healthy bone (18) it is made possible that the regenerated bone produced through the regeneration of the bone defect point (2) has a shape which corresponds to the shape the bone had at the site to be regenerated when it was still healthy.

### PRIOR ART

The invention is based on a method of manufacturing a cap of a covering device for a bone defect site of the type in claim 1 and a device for covering and/or reconstructing a bone defect site of the type according to claim 13.

In bone surgery, for example in the reconstruction of bones in orthopaedic, neurosurgical or plastic surgery or in maxillary surgery, bone defect sites in the form of recesses or cavities in the endogenous bone tissue are often filled with bone formation material. As a rule the bone formation material consists of a mixture of synthetic bone replacement material (e.g. hydroxylapatite granules) and endogenous bone particles. So that osseous growth through the bone formation material essentially exclusively takes place from the bone side, the recess is, as described in patent DE 43 02 708 C2, covered with a covering membrane. The covering membrane is fixed to the endogenous bone with fastening nails wherein, as the covering membrane is made of flexible material, fastening requires the utmost skill on the part of the surgeon.

In order to overcome this drawback of a lack of support function of the covering membrane, in patent U.S. Pat. No. 4,816,339 a covering membrane is described which consists of several layers, wherein these layers are not made of resorbable membrane material. Here, after healing of the bone defect it may be necessary to carry out a second intervention to remove exogenous material.

In patent DE 10 2005 039 382 B4 a biodegradable hollow body, in particular with a hollow cylindrical or conical cylindrical shape, is proposed. In its walls the hollow cylinder has a plurality of openings through which blood can be taken up thereby making the formation of endogenous bone possible. A disadvantage of this is that to insert the hollow body a cylindrical bored hole must be produced in the existing bone by a drill.

In unexamined and published patent DE 10 2006 047 054 A1 an implant bearing is proposed which is characterised by an accurate fit and stability, so that the treating doctor can simply handle and implant it. The implant bearing, made of hydroxylapatite, which to protect the mucous membranes from mechanical effects and to protect the implant bearing from growing in tissue from the side of the mucous membrane has a thin membrane, more particularly made of resorbable material on the side facing the mucous membrane, is produced using a build-up manufacturing process so that the material quality has a “gradient structure” in the form of a density which decrease inwards. On the side facing the bone a construction with an, in particular, porous structure and on the outer side of the implant bearing, at which a structure for holding a tooth implant and/or denture is located, a compact structure is envisaged.

Furthermore in unexamined and published documents DE 198 30 992 A1, DE 10 2005 060 761 A1, DE 42 26 465 A1, WO 01/91818 A1, DE 10 2005 041 412 A1, DE 10 2006 047 054 A1, US 2011/0151400 A1, and WO 2006/051401 A2 describe devices for a bone defect site, wherein all of these solutions have the drawback that in addition to the bone defect site, they also affect present healthy bones.

In unexamined and published document DE 10 2011 011 191 A1 a method of manufacturing a cap of a covering device for a bone defect site is described in which in a first processing step a data set is recorded which represents the affect bone defect site three-dimensionally. The data set is then used for planning the cap. After conversion of the planning of the cap into a planning data set, the planning data set is supplied to a computer-controlled manufacturing process so that through this the cap is made of dimensionally-stable material, wherein its wall facing the bone defect or its wall facing away from the bone defect corresponds to the shape of the regenerated bone. A disadvantage of this is that the shape of the regenerated bone is calculated on the basis of the bone defect site present in a first processing stage so that the shape of the regenerated bone is based on a speculative assumption of how the regenerated bone should look. However, it cannot be ruled out that the calculated shape of the regenerated bone will differ from the desired ideal shape of the regenerated bone (intended condition or original condition).

### THE INVENTION AND ITS ADVANTAGES

The method according to the invention of manufacturing a cap for a covering device for a bone defect site with the characterising features of claim 1 and the device according to the invention for covering and/or reconstructing a bone defect site, wherein the term “bone defect site” denotes a site of a (diseased, deformed, injured, changed through the ageing process, through degeneration (e.g. after dental extraction, tumour etc.) or changed in volume) bone (e.g. hip, spinal column, head, jaw etc.) of a human or animal which deviates from the shape and/or the volume of a health bone, with the characterising features of claim 13, have the advantage that through a comparison of a first data set representing the affected bone defect site in the actual condition with a second data set representing the intended condition of a bone regenerated at the bone defect site, wherein the second data set is calculated or was recorded at a time when the bone at the now to be regenerated site was still a healthy bone, it is made possible that the regenerated bone, produced through regeneration of the bone defect site, has a shape that corresponds to the shape the bone at the regenerated site had when it as still healthy The second data set of the healthy bone can thus be based on an actual measurement and not, as known through the prior art, on a calculation of the shape of the bone to be regenerated. According to the invention preservation of the intended condition can therefore take place. This means that a data set has been produced from a healthy bone in order that, if required, (possibly years or decades later) this data set can be used if a bone defect has occurred on this documented healthy bone in order together with the data set recording the current bone defect to initiate a suitable therapeutic treatment method which, for example, comprises the manufacturing of a cap or a covering device for the bone defect based on the first data set and the second data set.

According to an advantageous embodiment of the method according to the invention of manufacturing a cap of a covering device for a bone defect site, in which preferably the computer assisted design (CAD) of the cap is combined with computer assisted manufacturing (CAM) into CAD/CAM, so that a design model of the cap developed on the computer is transferred directly in electronic form to manufacturing, consisting of the following processing stages:
recording of first data set representing the affected bone defect site in the actual condition,
a comparison of the first data set with a second data set representing the intended condition of a bone regenerated at the bone defect site, wherein the second data set is calculated or was recorded at a time when the bone at the now to be regenerated site was still a healthy bone, and
Use of the first data set and the second data set for planning the cap, which has a wall (surface) facing away from the bone defect and a wall (surface) facing the bone defect, and is, if applicable, fixable to a bone with at least one fixing means,
Conversion of the planning of the cap into a planning data set and
supplying the data set to a manufacturing process, in particular a computer-controlled manufacturing process, in which the cap is made of a dimensionally stable material and its wall (surface) facing the bone defect and its wall (surface) facing away from the boned defect correspond to the shape of the regenerated bone in the nominal state, wherein during and/or after manufacturing of the cap at least one nominal breaking point is provided on the cap,
   wherein the recording of the first data represents the affected bone defect site in its three-dimensionality and/or the recording of the second data set represents the shape of the still healthy bone in its three-dimensionality.

According to an additional advantageous embodiment of the method according to the invention of manufacturing a cap of a covering device for a bone defect site, the first data set which represents the actual condition and/or the second data set which represents the nominal condition are recorded by means of at least one imaging process.

According to an additional advantageous embodiment of the method according to the invention of manufacturing a cap of a covering device for a bone defect site, the first data set and/or the second data set are recorded by means of at least one process which allows a bone to be shown three-dimensionally. More particularly, the first data set and/or the second data set are recorded by means of tomography, computer tomography, digital volume tomography, sonography etc.

According to an additional advantageous embodiment of the method according to the invention of manufacturing a cap of a covering device for a bone defect site, the data set of the healthy bone is recorded after the healthy bone has matured. In this way it is possible that, if need be, the ideal state (nominal condition) of the bone is documented so that it is known what any subsequently to be regenerated bone should look like. In humans recording of the healthy bone data set should preferably take place between ages of 18 and 25 years. Of course it is also conceivable that in the mature state of the bones several healthy bones or the skeleton of the person or animal is/are recorded, documented and/or stored. It would also be conceivable to at least partially produce a cap at the time of recording of the healthy bone.

According to an additional advantageous embodiment of the method according to the invention of manufacturing a cap of a covering device for a bone defect site, the data set of the healthy bone is stored on a storage medium for later use (preserved).

According to an additional advantageous embodiment of the method according to the invention of manufacturing a cap of a covering device for a bone defect site, the cap is produced by milling during the manufacturing process.

According to an additional advantageous embodiment of the method according to the invention of manufacturing a cap of a covering device for a bone defect site, during and/or after manufacturing of the cap at least one fastening device is provided on the cap for the insertion of at least one implant. The fastening device can, for example, be in the form of a recess.

According to an embodiment of the method according to the invention for manufacturing a cap of a covering device for a bone defect site which is advantageous in this respect, at least one fastening device (e.g. a recess) is exposed by removing a part of the cap, which before removal is connected to the remaining part of the cap by way of at least one nominal breaking point. The time of exposure of the fastening device can be before or after arranging the covering device on the bone defect site.

According to an additional advantageous embodiment of the method according to the invention of manufacturing a cap of a covering device for a bone defect site, during and/or after manufacturing of the cap at least one positioning means is arranged on the cap which serves to position the cap on a healthy bone adjoining the bone defect site and which has a wall (surface) facing away from the healthy bone and a wall (surface) facing the health bone and at least partially corresponding therewith.

According to an advantageous embodiment of the method according to the invention of manufacturing a cap of a covering device for a bone defect site, at least one nominal breaking point is arranged between the cap and a positioning means.

According to an additional advantageous embodiment of the method according to the invention of manufacturing a cap of a covering device for a bone defect site, after manufacturing of the cap a cleaning and/or sterilisation process is carried out.

According to an advantageous embodiment of the device according to the invention for covering and/or reconstructing a bone defect, comprising a cap which has a wall (surface) facing the bone defect and a wall (surface) facing away from the bone defect and possibly at least one fixing means for fixing the cap to a bone, wherein the cap is made of a dimensionally stable material which is at least partially (at the edge) in contact with the bone, and a wall of the cap facing the bone defect or a wall of the cap facing away from the bone defect corresponding to the shape of the regenerated bone and the cap has at least one nominal breaking point, the cap and/or the fixing means are at least partially made of a biocompatible material. The biocompatible material can be biotolerant, bio-inert and/or bioactive. The nominal breaking point allows the cap to be divided into a least two parts so that, if removal of the cap after bone generation is desired, it can be easily removed.

According to another advantageous embodiment of the device according to the invention the material is of organic and/or inorganic origin. This can be an autogenic, syngenic, allogenic, xenogenic, synthetic or alloplastic material.

According to another advantageous embodiment of the device according to the invention the cap and/or the fixing means at least partially consist of a biodegradable material.

According to another advantageous embodiment of the device according to the invention the cap and/or the fixing means can at least partially consist of a resorbable material. Advantageously the resorption time of the rigid shell can be controlled through its resorption gradient and/or the resorption time can also be less than six months so that the implant can be inserted within a short time frame. Preferably resorbable metals or alloys, in particular magnesium or magnesium alloys are used. The 3D models (e.g. the cap and/or the fixing means) are preferably constructed using the laser melting process wherein a 3D printer is preferably used.

According to another advantageous embodiment of the device according to the invention the cap and/or the fixing means at least partially consist of a polymer or a polymer compound.

According to another advantageous embodiment of the device according to the invention the cap and/or the fixing means at least partially consist of polyactide. Polyactides are built up of many lactic acid molecules chemically bonded to each other and belong to the polymers. The advantage of polylactide plastics, also known as polylactic acids (PLT) is that through the supply of heat they are deformable plastics and are biocompatible.

According to another advantageous embodiment of the device according to the invention the cap has a varying wall thickness.

According to an advantageous embodiment of the device according to the invention in this respect the wall thickness should be at least 0.2 mm, preferably 0.5 mm, but at least so much that dimensional stability of the mould shell is brought about.

According to an additional advantageous embodiment of the device according to the invention, the fixing means is a pin, a screw, a nail and/or a bone adhesive. In order to protect healthy bone the fixing means is preferably arranged in the region of the bone defect site.

According to another advantageous embodiment of the device according to the invention the cap has milling (boring for the fixing means).

According to an embodiment of the device according to the invention which is advantageous in this respect, the milling corresponds to the fixing means.

According to another advantageous embodiment of the device according to the invention the wall facing the bone defect has undergone surface conditioning.

According to an embodiment of the device which is advantageous in this respect the surface can have a micro-structuring, pores, osteoblast attractants, means for promoting bone growth and/or bone replacement means containing BMP.

According to another advantageous embodiment of the device according to the invention the cap has at least one opening. This means that the cap does not have to have a closed wall. Through a plurality of openings the cap can, at least in parts, have a net-like structure, wherein the wall of the net-like structure facing away from the wall or the wall of the net-like structure facing the wall corresponds to the shape of the regenerated bone.

According to another advantageous embodiment of the device according to the invention the cap has at least one fastening device (e.g. a recess) for at least one insertable implant.

According to an embodiment of the device according to the invention which is advantageous in this respect, at least one fastening device (e.g. a recess) is covered by a part of the cap which by way of at least one nominal breaking point is connected to the remaining part of the cap.

According to an additional advantageous embodiment of the device according to the invention, for positioning the cap on a healthy bone adjoining the bone defect site at least positioning means is provided which has a wall facing away from the healthy bone and a wall facing the healthy bone and at least partially corresponding therewith.

According to an embodiment of the device according to the invention which is advantageous in this respect, at least one nominal breaking point is arranged between the cap and a positioning means. In this way a positioning means applied to the healthy bone and thus possibly disruptively projecting therefrom, e.g. after fixing of the cap and/or after regeneration of the bone at the bone defect site can be removed from the remaining cap.

Through the method according to the invention a device according to the invention for covering and/or reconstructing a bone defect site can be created, the cap and/or fixing means of which, for example, are made of a material of organic and/or inorganic origin. This can also be a synthetic material and/or a material or autogenic, synergenic, allogenic and/or xenogenic, alloplastic, human and/or animal origin. The human, animal or synthetic matrix can be of a shape through which the area located between the bone and the required shape of the regenerated bone is fully or almost filled. For this, a bone block, for example, is taken from the donor (autologous or non-related donor) which may then be modelled by CAD/CAM.

Further advantages and advantageous developments of the invention become apparent from the following description, drawing and claims.

### DRAWING

Examples of embodiment of the subject matter of the invention are set out in the drawing and will be explained in more detail below. Here:

FIG. 1 shows device according to the invention for covering and/or reconstructing a bone defect site

FIG. 2 shows a differently shaped device according to the invention for covering and/or reconstructing a bone defect site

FIG. 3 shows a differently shaped device according to the invention for covering and/or reconstructing a bone defect site

FIG. 4 shows a differently shaped device according to the invention for covering and/or reconstructing a bone defect site

FIG. 5 shows as section for a cap

FIG. 6 to 8 show various views of a cap with positioning means and

FIG. 9 shows a cap arranged on the bone defect site.

### DESCRIPTION OF THE EXAMPLES OF EMBODIMENT

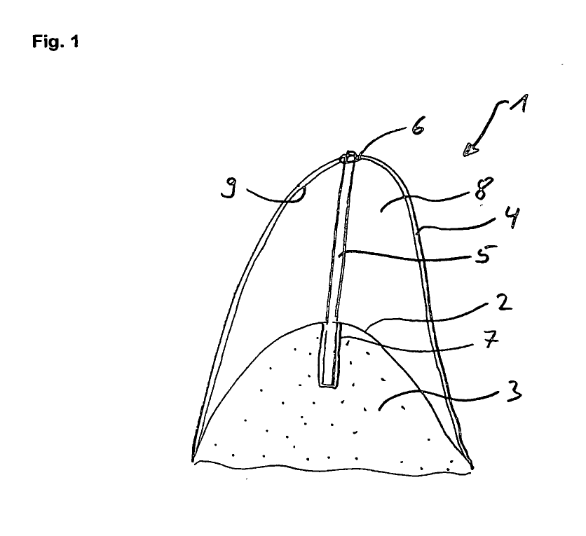

FIG. 1 shows a view of a device 1 according to the invention for covering and/or reconstructing a bone defect site 2 (bone defect) of a bone, in particularly a jaw bone 3. The device 1 comprises a cap 4, which is in one layer, and a fixing means 5, which in FIG. 1 is shown as a pin 1 arranged in the bone defect site 2. The cap 4 is made of a dimensionally stable material so that it is self-supporting and no additional support is required. For fixing the cap 4 (moulding shell, rigid shell) the fixing means 5 is pushed through a boring 6 in the cap 4 and is then introduced into the boring 7 in the jaw bone 3. Subsequent fixation of the cap 4 preferably takes place by way of ultrasonic welding. In the ultrasonic welding an ultrasound generator preferably produces a precisely defined frequency which is bundled via a sonotrode. After application of the resorbable fixing means 5 (pin) to a drilled hole (boring 7) in the bone, a produced oscillation fluidises the pin surfaces at their edges which brings about sliding of the pin into the boring. Through changing the state of the generator the pin also penetrates into the osseous cavities which a conventional bone screw cannot usually reach so that a high initially stability is achieved. The pin 4 also combines with the cap 4 and through a blocking mechanism ensures a stable three-dimensional structure. During the ultrasonic welding the fixing means 5 is thus softened so that it combines with the jaw bone 3 and the cap 4. Through the affixed cap 4 a sealed-off inner space 8 is formed between the jaw bone 3 and the cap 4 which is filled through the regeneration of the bone and/or through the introduction of a material of organic and/or inorganic origin, which can also be an autogenic, syngenic, allogenic, xenogenic, synthetic and/or alloplastic material, so that the regenerated bone or the introduced material corresponds to the shape of the wall 9 (surface) of the cap facing bone defect site 2. In order to accelerate the regeneration process of the jaw bone 3, the wall 9 of the cap 4 facing the bone defect can have undergone surface conditioning (e.g. micro-structuring, pores, osteoblast attractants, means for promoting bone growth and/or bone replacement means containing BMP).

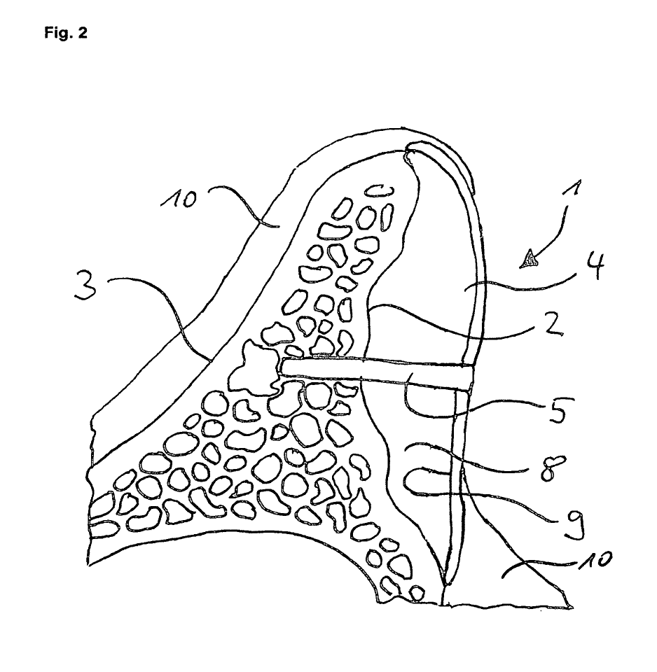

FIG. 2 shows a view of a differently shaped device 1 according to the invention for covering and/or reconstructing a bone defect site 2 (bone defect) of a bone, in particular a jaw bone 3. In this figure the gums 10 are also indicated.

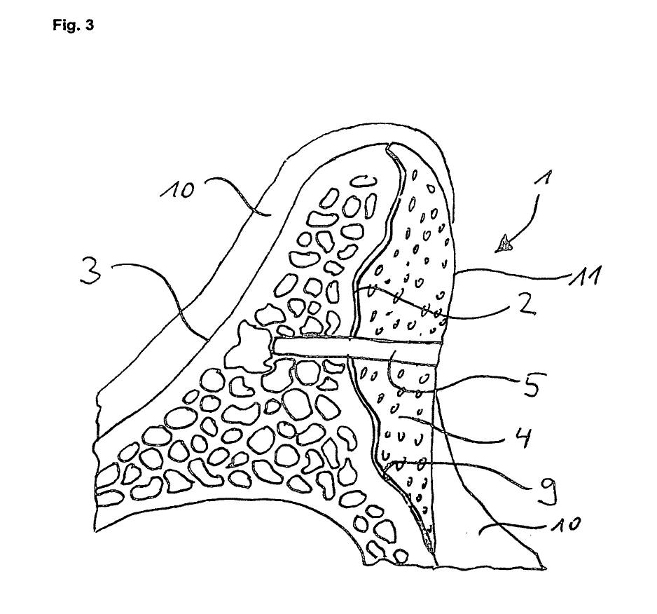

FIG. 3 shows a view of a differently shaped device 1 according to the invention for covering and/or reconstructing a bone defect site 2 (bone defect) of a bone, in particularly a jaw bone 3. In this figure the cap 4 is in the form of a moulded body, e.g. made of human or animal bone, and has a wall (surface) 9 facing the bone defect, which is adapted to the relief of the bone defect site 2, and a wall 11 (surface) facing away from the bond defect site 2 which corresponds to the shape of the regenerated bone.

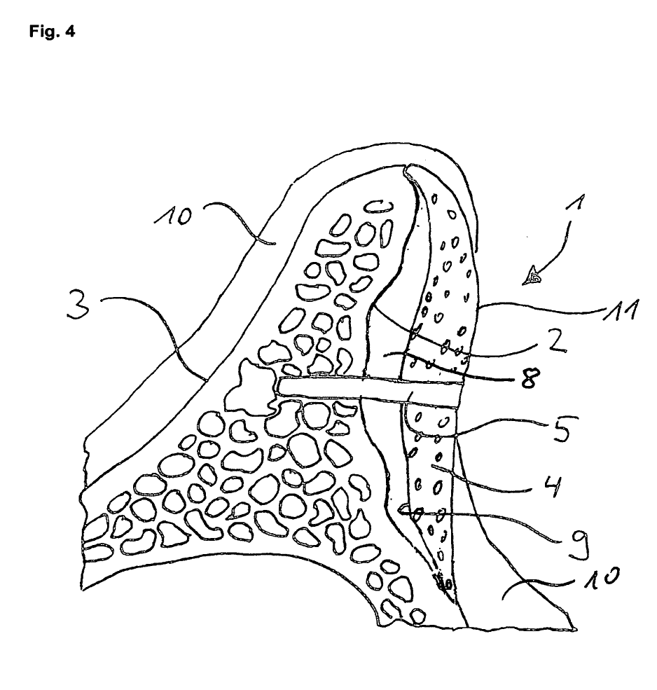

FIG. 4 shows a view of a differently shaped device 1 according to the invention for covering and/or reconstructing a bone defect site 2 (bone defect) of a bone, in particularly a jaw bone 3. In this figure the cap 4 is in the form of a moulded body, e.g. made of human or animal bone, and has a wall 9 facing the bone defect and a wall 11 facing away from the bone defect site which corresponds to the shape of the regenerated bone.

Between the wall 9 and the bone defect 2 is an internal space 8 which is filled through the regeneration of the bone and/or through the introduction of autogenic, syngenic, allogenic, xenogenic, synthetic and/or alloplastic material.

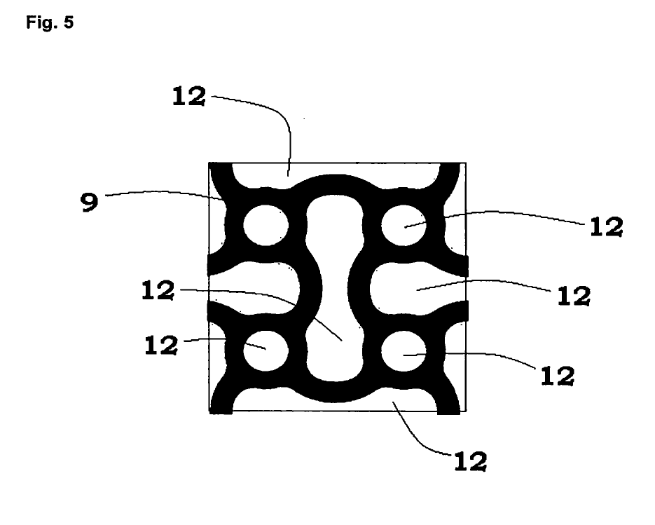

FIG. 5 shows a section of a cap 4 the wall 9 of which facing the bone defect has an opening 12 through which a net-like structure is formed.

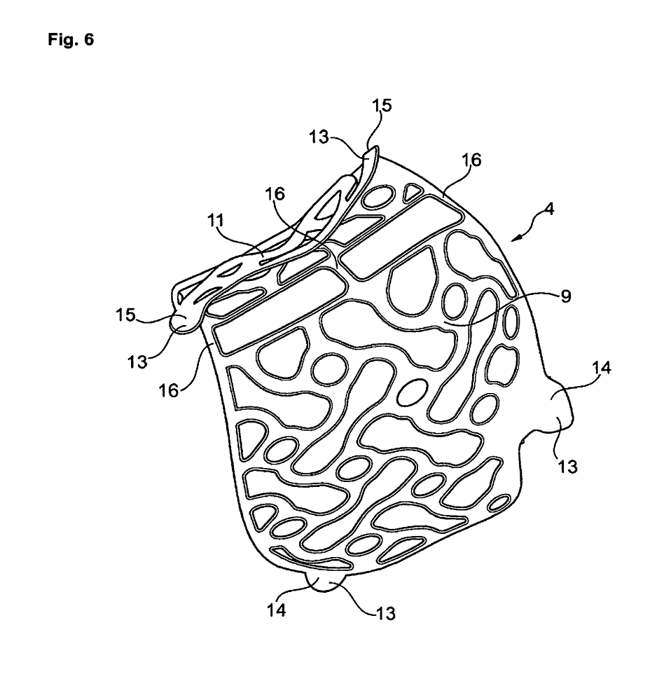

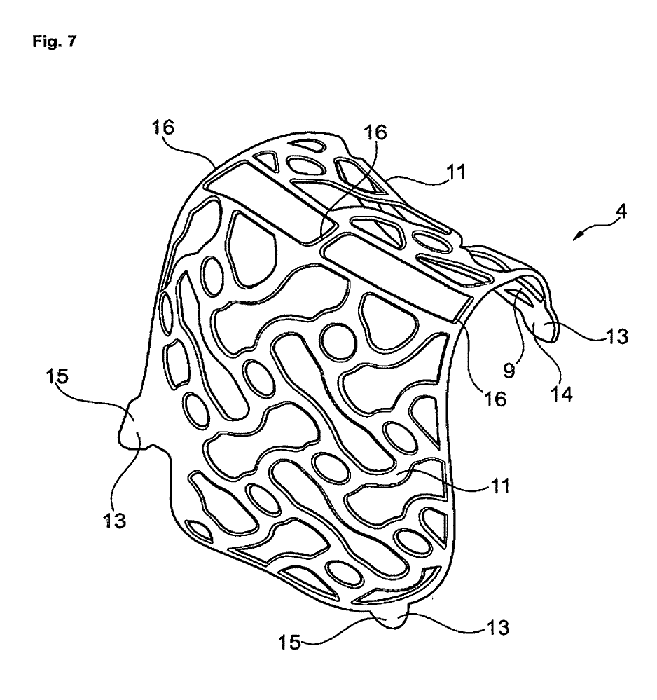

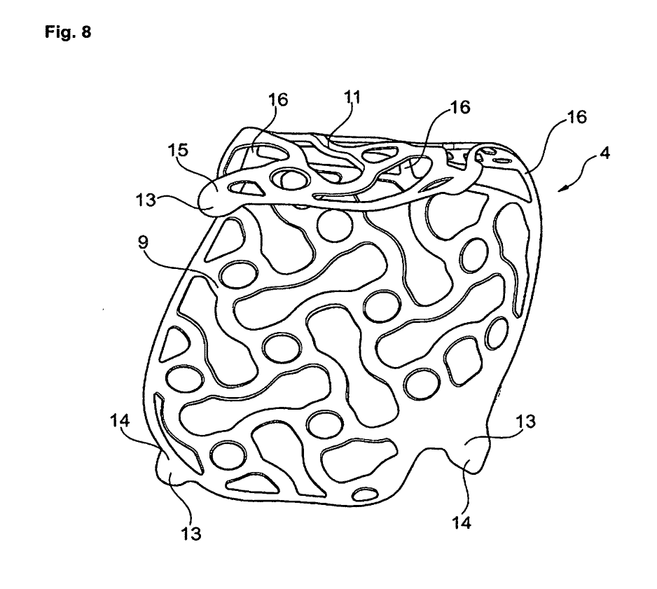

FIGS. 6 to 8 shows various views of a cap 4 which has a wall 9 facing a bone defect site and a wall 11 facing away from the bone defect site, with positioning means 13 which have a wall 14 facing a healthy bone and a wall 15 facing away from the healthy bone. When the cap 4 is correctly arranged on the bone defect site the walls 14 facing a healthy bone are in contact with the healthy bone through which by way of the positioning means 13 a perfect fit of the cap 4 is guaranteed. In order to be able to easily remove the cap 4 after bone regeneration it has a nominal breaking point 16 wherein after cutting through this it can be divided into two parts for removal.

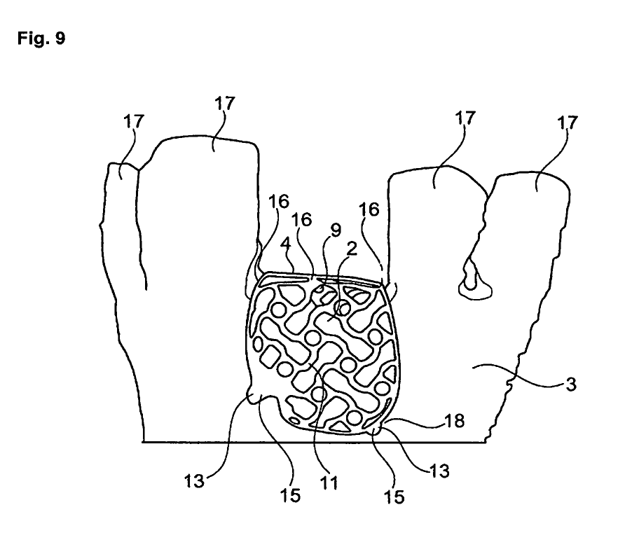

FIG. 9 shows a cap 4 arranged on a bone defect site 2 of jaw bone 3 having teeth 17 which is shown as an excerpt. Through this it can be seen that the cap 4 is preferably only arranged in the area of the bone defect site 2 of the jaw bone 3 so that it neither bridges nor contacts a healthy bone 18. Only the positioning means 13 arranged on the cap 4 are thus in contact with the healthy bone 18.

Shown in FIG. 6 to 8 is a cap, the wall 9 of which facing the bone defect corresponds to the shape of the regenerated bone. It is also conceivable for the positioning means 13 to be arranged on the cap 4 in such a way that its wall 11 facing the bone defect corresponds to the shape of the regenerated bone. This could be brought about, for example through arranging the positioning means 13 on the wall 11 of the cap 4 facing away from the bone defect.

All the features set out here can be essential to the invention either alone or also in any combination with each other.

### LIST OF REFERENCE NUMBERS

1 Device

2 Bone defect site

3 Jaw bone

4 Cap

5 Fixing means

6 Boring

7 Boring

8 Interior space

9 Wall

10 Gums

11 Wall

12 Opening

13 Positioning means

14 Wall

15 Wall

16 Nominal breaking point

17 Tooth

18 Healthy bone

1: Process for manufacturing a cap (4) of a covering device for a bone defect site (2) comprising the following processing stages:
recording of a first data set which represents the affected bone defect site (2) in the actual condition
comparison of the first data set with a second data set which represents the nominal condition of a bone regenerated at the bone defect site (2), wherein the second data set is produced through calculation or is recorded at a time at which the bone at the now to be regenerated site was still a healthy bone (18),
use of the first data set and the second data set for planning the cap (4), which comprises a wall (11) facing away from the bone defect and a wall (9) facing the bone defect and, if need be, can be fixed on a bone with at least one fixing means (5),
conversion of the planning of the cap (4) in to planning data set and
transferring the planning data set to a manufacturing process, more particularly a computer-controlled manufacturing process in which the cap (4) is made of a dimensionally stable material and its wall (9) facing the bone defect or its wall (11) facing away from the bone defect site represents the shape of the regenerated bone in the nominal condition, wherein during and/or after manufacturing of cap (4) at least one nominal breaking point is arranged on the cap (4).

2: Process according to claim 1 wherein the recording of the first data set represents the affected bone defect site (2) in its three-dimensionality and/or the recording of the second data set represents the form of the still health bone (18) in its three-dimensionality.

3: Process according to claim 1 wherein recording of the first data set and/or the recording of the second data set takes place by way of at least one imaging process.

4: Process according to claim 1 wherein the recording of the first data set and/or the recording of the second data set takes place by means of a process which allows three-dimensional representation of a bone.

5: Process for manufacturing a cap (4) according to claim 1 wherein the recording of the data set of the healthy bone (18) takes place after the healthy bone (18) has matured.

6: Process for manufacturing a cap (4) according to claim 5 wherein the data set of the healthy bone (18) is stored on a storage medium for later use.

7: Process for manufacturing a cap (4) according to claim 1 wherein in the manufacturing process the cap (4) is produced by way of milling.

8: Process for manufacturing a cap (4) claim 1 wherein during and/or after manufacturing of the cap (4) at least one fastening device is arranged on the cap (4) for fastening at least one implant to be inserted.

9: Process for manufacturing a cap (4) according to claim 8 wherein at least one fastening device is exposed by removing a part of the cap (4), which before being removed is connected by means of at least one nominal breaking point to the remaining part of the cap (4).

10: Process for manufacturing a cap (4) according to claim 1 wherein during and/or after the manufacturing of the cap (4) at least one positioning means (13) is arranged on the cap (4) which serves to position the cap (4) on a healthy bone (18) adjoining the bone defect site (2) on the cap (4) and which has a wall (15) facing away from the healthy bone (18) and a wall (14) facing the health bone (18) and at least partially corresponding therewith.

11: Process for manufacturing a cap (4) according to claim 10 wherein at least one nominal breaking point (16) is arranged between the cap (4) and a positioning means (13).

12: Process for manufacturing a cap (4) according to claim 1 wherein after manufacturing of the cap (4) a cleaning and/or sterilization process is carried out.

13: Device (1) for covering and/or reconstructing a bone defect site (2)
with a cap (4) which as a wall (11) facing away from the bone defect site and a wall (9) facing the bone defect,
with, if need be, a fixing means (5) for fixing the cap (4) to a bone,
wherein the cap (2) is made of a dimensionally stable material which is at least partially in contact with the bone, and a wall (9) of the cap (2) facing the bone defect or a wall (11) of the cap (4) facing away from the bone defect corresponds to the shape of the regenerated bone,
wherein
the cap (4) has at least one nominal breaking point (16).

14: Device (1) according to claim 13 wherein the cap (4) and/or the fixing means (5) is/are at least partially made of a biocompatible material.

15: Device (1) according to claim 13 wherein the material is of organic and/or inorganic origin.

16: Device (1) according to claim 13 wherein at least one of the cap (4) and the fixing means (5) at least partially comprises a biodegradable material.

17: Device (1) according to claim 13 wherein at least one of the cap (4) and the fixing means (5) at least partially comprises a resorbable material.

18: Device (1) according to claim 13 wherein at least one of the cap (4) and the fixing means (5) comprises at least partially a polymer or a polymer compound.

19: Device (1) according to claim 13 wherein at leasdt one of the cap (4) and the fixing means (5) comprises at least partially polylactide.

20: Device (1) according to claim 13 wherein the cap (4) has a constant or a varying wall thickness.

21: Device (1) according to claim 20 wherein the wall thickness is at least 0.2 mm.

22: Device (1) according to claim 13 wherein the fixing means (5) is a pin, a screw, a nail and/or a bone adhesive.

23: Device (1) according to claim 13 wherein the cap (4) comprises at least one milling.

24: Device (1) according to claim 23 wherein the milling corresponds with the fixing means (5).

25: Device (1) according to claim 13 wherein the wall (9) facing the bone defect has undergone surface conditioning.

26: Device (1) according to claim 25 wherein the surface conditioning has a micro-structuring, pores, osteoblast attractants, means for promoting bone growth and/or bone replacement means containing BMP.

27: Device (1) according to claim 13 wherein the cap (4) comprises at least one opening.

28: Device (1) according to claim 13 wherein the cap (4) has at least one fastening device for at least one implant to be inserted.

29: Device (1) according to claim 28 wherein at least one fastening device is at least partially covered by a part of the cap (4) which is connected to the remaining part of the cap (4) by means of at least one nominal breaking point.

30: Device (1) according to claim 13 wherein for positioning the cap (4) on a health bone (18) adjoining the bone defect site (2) at least one positioning means (13) is arranged on the cap (4) which has a wall (15) facing away from the healthy bone and a wall (14) facing the healthy bone (18) and at least partially corresponding therewith.

31: Device (1) according to claim 30 wherein at least one nominal breaking point (16) is arranged between the cap (4) and a positioning means (13).

32: Device (1) for covering and/or reconstructing a bone defect site (2)
with a cap (4) which as a wall (11) facing away from the bone defect site and a wall (9) facing the bone defect,
with, if need be, a fixing means (5) for fixing the cap (4) to a bone,
wherein the cap (2) is made of a dimensionally stable material which is at least partially in contact with the bone, and a wall (9) of the cap (2) facing the bone defect or a wall (11) of the cap (4) facing away from the bone defect corresponds to the shape of the regenerated bone,
wherein
the cap (4) has at least one nominal breaking point (16); and wherein the cap (4) is manufactured in accordance with the process for manufacturing a cap (4) according to claim 1.
